(19) United States
(12) Patent Application Publication
(10) Pub. No.: US 2026/0069848 A1
(43) Pub. Date: Mar. 12, 2026

(54) Catheter Assembly with Near-Patient Connector Having Improved Back-Pressure Capability

(71) Applicant: Becton, Dickinson and Company, Franklin Lakes, NJ (US)

(72) Inventors: Weston F. Harding, Lehi, UT (US); Jonathan Karl Burkholz, Salt Lake City, UT (US); Dorien Gemma Butter, Millcreek, UT (US); Erica E. Neumann, Sandy, UT (US); Michael Glen Beeman, Salt Lake City, UT (US)

(21) Appl. No.: 19/264,048

(22) Filed: Jul. 9, 2025

Related U.S. Application Data
(60) Provisional application No. 63/672,271, filed on Jul. 17, 2024.

(51) Int. Cl. A61M 39/06; A61B 5/15; A61B 5/153; A61M 39/00; A61M 39/02

(52) U.S. Cl. CPC A61M 39/0606; A61B 5/150221; A61B 5/150992; A61B 5/153; A61M 2039/0018; A61M 2039/0202; A61M 2039/0258; A61M 2039/064; A61M 2039/0646; A61M 2039/066; A61M 2039/0673

(57) ABSTRACT

A catheter assembly is provided that includes a catheter having a catheter distal end and a catheter proximal end, a catheter adapter coupled to the catheter proximal end and including a lumen in fluid communication with the catheter and an adapter port in fluid communication with the lumen, a near-patient adapter connected to the adapter port via an extension tube and including a distal end and a proximal end, and a needle-free connector coupled to the proximal end of the near-patient adapter. The needle-free connector includes a main body defining an inner lumen and including a distal port and a proximal port, a split septum valve retained within the proximal port and extending into the inner lumen, and a pressure resistance-increasing feature incorporated into or provided adjacent to the split septum valve configured, to increase a pressure capability of the needle-free connector.

### CROSS-REFERENCE TO RELATED APPLICATION

The present application claims priority to U.S. Provisional Patent Application No. 63/672,271 entitled “Catheter Assembly with Near-Patient Connector Having Improved Back-Pressure Capability” filed Jul. 17, 2024, the entire disclosure of which is hereby incorporated by reference.

### BACKGROUND OF THE INVENTION

### Field of the Invention

The present invention is directed to a catheter assembly that provides for insertion of and access to an indwelling catheter and, more particularly, to a near-patient connector for the catheter assembly having an improved back-pressure capability.

### Background of the Invention

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

In some instances, the VAD is part of a larger catheter system or assembly, where tubing extends out from an adapter or connector of the VAD to provide additional flexibility for medical professionals in terms of administering and/or withdrawing fluids from the VAD—with such tubing being part of an extension set that is integrated with the VAD (an integrated catheter system) or provided separate from the VAD (a non-integrated catheter system), according to various embodiments. One example of an existing integrated catheter systems from Becton, Dickinson and Company (“BD”) is the BD Nexiva™ Closed Peripheral IV Catheter System with NearPort™ IV Access, as a non-limiting example.

As indicated above, VADs such as the BD Nexiva™ Closed Peripheral IV Catheter System with NearPort™ IV Access may be used for a fluid infusion and/or blood withdrawal, with it recognized that such fluid infusion and/or blood withdrawal may be performed via one or more external devices or systems useable with the VAD to aid in fluid infusion and/or blood withdrawal directly from the indwelling catheter of the VAD. VADs such as those mentioned above may often include multiple connectors or access ports by which external devices may be connected to the catheter system, in order to provide for the administering and/or withdrawing fluids from the VAD. Such connectors may generally be characterized as a “near port” connector and a “far port” connector. The near port connector may be ideal for connection of a blood draw device such as the PIVO™ from Becton, Dickinson and Company, which is configured to advance a flexible, internal probe or flow tube into/through the indwelling catheter of the catheter system. The far port connector may be ideal for connection of a fluid delivery device thereto, for an intravenous infusion via a gravity flow drip, syringe push, or saline flush, as non-limiting examples.

As indicated above, when using a device for fluid delivery with a VAD, it is common for the device to be connected to the far port of the VAD. Under normal use, the near port connector and its access valve is able to hold pressure within the system and accommodate the fluid delivery without issue, but there are some situations where the pressure can exceed the capabilities of the near port luer access valve, and a leak may occur. For example, when a bolus of medication is given with a syringe attached to the far port (1) at a rate higher than the catheter can accommodate and/or (2) when the catheter is occluded, a high (back) pressure may be applied against the near port luer access valve, which can result in a leak through the valve.

Accordingly, a need exists for VADs that include a near-patient access valve or port having an improved back-pressure capability, such that the near-patient access valve or port is better able to hold or withstand higher internal pressure before leaking.

### SUMMARY OF THE INVENTION

Provided herein is a catheter assembly that includes a catheter having a catheter distal end and a catheter proximal end, a catheter adapter coupled to the catheter proximal end and including a lumen in fluid communication with the catheter and an adapter port in fluid communication with the lumen, a near-patient adapter connected to the adapter port via an extension tube and including a distal end and a proximal end, and a needle-free connector coupled to the proximal end of the near-patient adapter. The needle-free connector includes a main body defining an inner lumen and including a distal port and a proximal port, a split septum valve retained within the proximal port and extending into the inner lumen, and a pressure resistance-increasing feature incorporated into or provided adjacent to the split septum valve configured, to increase a pressure capability of the needle-free connector.

In some embodiments, the pressure resistance-increasing feature comprises a circumferential bump formed on an inner surface of main body and extending radially inward into the inner lumen, at a location adjacent the split septum valve.

In some embodiments, the circumferential bump increases a compressive force between an external male connector and the split septum valve, when the external male connector is inserted through a slit of the split septum valve and into the inner volume of the main body.

In some embodiments, the external male connector comprises one of a male luer connector and a blunt cannula.

In some embodiments, the pressure resistance-increasing feature comprises a secondary split septum valve positioned within the inner lumen and distal to the split septum valve.

In some embodiments, the split septum valve is configured to receive a blunt cannula of an external male connector, and the pressure resistance-increasing feature comprises a reduced-length slit formed in the split septum valve.

In some embodiments, the main body further includes a side port located between the distal port and the proximal port, the side port in fluid communication with the inner lumen, the side port comprising a luer connection.

In some embodiments, the main body defines a bowl forming part of the inner lumen, the bowl extending distally past the split septum valve, and wherein the side port enters into the bowl distal to the split septum valve.

In some embodiments, the catheter assembly further comprises an extension set coupled to the side port.

In some embodiments, the pressure resistance-increasing feature comprises a bellows-shaped tube provided on the split septum valve that extends distally from a slit formed in the split septum valve, the bellows-shaped tube configured to receive an external male connector therein when engaged with the split septum valve.

In some embodiments, the external male connector comprises a blunt cannula, and wherein the bellows-shaped tube comprises a lubricant applied onto an internal surface thereof, to reduce friction during insertion of the blunt cannula into the bellows-shaped tube.

In some embodiments, the distal port comprises a male luer connection and the proximal port comprises a female luer connection.

Also provided herein is a catheter assembly useable with a fluid transfer device having a blunt cannula distal connection. The catheter assembly includes a catheter having a catheter distal end and a catheter proximal end and a catheter adapter coupled to the catheter proximal end and including a lumen in fluid communication with the catheter, the catheter adapter including an adapter port in fluid communication with the lumen. The catheter assembly also includes a near-patient adapter comprising a distal end and a proximal end, the distal end coupled to the adapter port via an extension tube, and a valve retained within the proximal end of the near-patient adapter, wherein the valve is configured to receive a blunt cannula connector of a fluid transfer device therein to fluidly connect the fluid transfer device with the inner lumen, with the blunt cannula having a diameter of 3 mm or less.

In some embodiments, the valve comprises a duckbill valve.

In some embodiments, the valve comprises a split septum valve.

Also provided herein is a system that includes a catheter having a catheter distal end and a catheter proximal end and a catheter adapter coupled to the catheter proximal end and including a lumen in fluid communication with the catheter, the catheter adapter including an adapter port in fluid communication with the lumen. The system also includes a near-patient adapter comprising a distal end and a proximal end, the distal end coupled to the adapter port via an extension tube. The system further includes a needle-free connector coupled to the proximal end of the near-patient adapter, with the needle-free connector comprising a main body defining an inner lumen and including a distal port and a proximal port, a split septum valve retained within the proximal port and extending into the inner lumen, and a pressure resistance-increasing feature incorporated into or provided adjacent to the split septum valve configured, to increase a pressure capability of the needle-free connector. The system still further includes a blood draw device coupled to the proximal port, via the split septum valve, the blood draw device comprising a flow tube configured to be advanced distally out from the blood draw device and into or through the catheter, to enable performing of a blood draw. The blood draw device comprises a distal male connector that engages the split septum valve, with the pressure resistance-increasing feature preventing a leak out from the needle-free connector during performing of the blood draw.

In some embodiments, the distal male connector comprises a lock including a blunt cannula configured to engage the split septum valve and a pair of clip arms configured to secure the lock to the main body of the needle-free connector.

In some embodiments, the distal male connector comprises a male luer connector.

In some embodiments, the needle-free connector is rated to accommodate an internal pressure of up to 300 psi.

### BRIEF DESCRIPTION OF THE DRAWINGS

FIG. 1 illustrates a system for facilitating blood draw and/or administering of fluids for a patient, in accordance with an aspect of the disclosure;

FIG. 2 is an isolated view of a catheter assembly included in the system of FIG. 1;

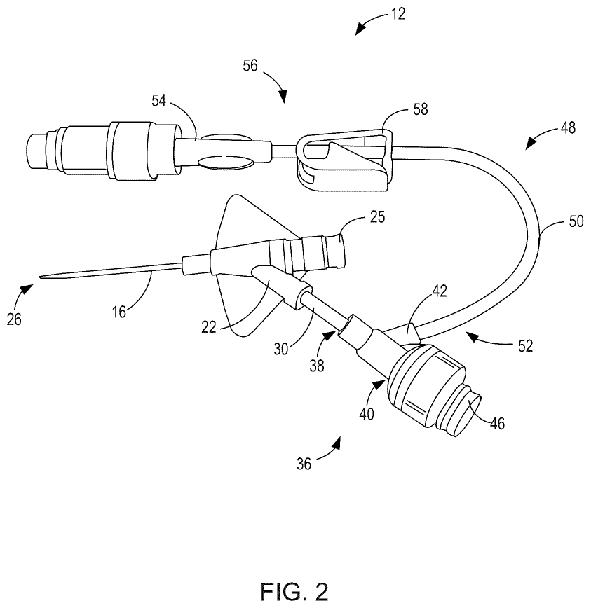

FIG. 3 is a perspective view of a needle-free connector included in the catheter assembly of FIG. 2, in accordance with an aspect of the disclosure;

FIG. 4A is a cross-sectional view of the needle-free connector of FIG. 3, with the split septum valve in a closed state;

FIG. 4B is a cross-sectional view of the needle-free connector of FIG. 3, with a male connection engaged therewith and the split septum valve in an open state;

FIGS. 5A and 5B are exploded perspective views of a needle-free connector included in the catheter assembly of FIG. 2, in accordance with another aspect of the disclosure;

FIG. 6 is a cross-sectional view of the needle-free connector of FIGS. 5A and 5B;

FIG. 7 is a cross-sectional view of a needle-free connector included in the catheter assembly of FIG. 2, in accordance with another aspect of the disclosure;

FIG. 8 is a perspective view of a needle-free connector included in the catheter assembly of FIG. 2, in accordance with an aspect of the disclosure;

FIG. 9 is a perspective view of a near-patient adapter included in the catheter assembly of FIG. 2, in accordance with another aspect of the disclosure; and

FIG. 10 is a perspective view of an access valve included in the near-patient adapter of FIG. 9.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

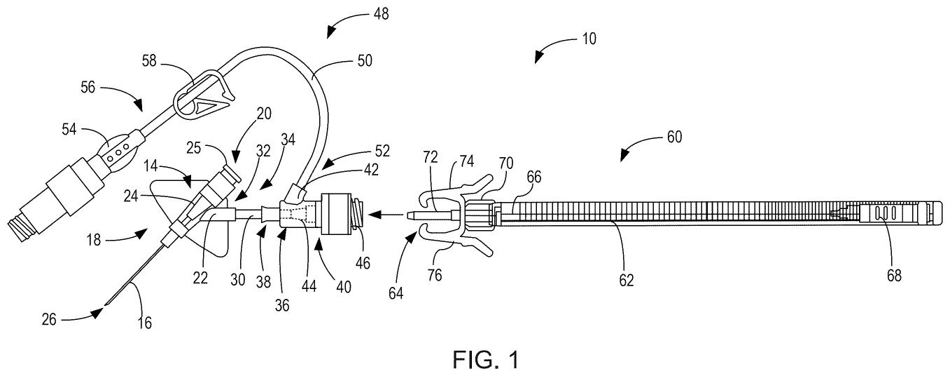

Referring to FIG. 1, shown is a non-limiting embodiment of a system 10 for facilitating blood draw and/or administering of fluids for a patient. As shown in FIG. 1, system 10 may include a catheter assembly 12 that provides access to the vasculature of a patient. According to embodiments of the disclosure, the catheter assembly 12 may have any of a number of suitable configurations, including being constructed/arranged as an integrated catheter assembly or a non-integrated catheter assembly that includes an extension set with a near-patient access port. Thus while FIG. 1 illustrates an integrated catheter assembly 12, it is recognized that aspects of the disclosure may be directed to other catheter assemblies. In FIG. 1, the catheter assembly 12 includes a catheter adapter 14 and associated catheter 16. The catheter adapter 14 may include a distal end 18 and a proximal end 20. In some embodiments, the catheter adapter 14 may include an additional adapter port 22 that may disposed between the distal end 18 and the proximal end 20 or disposed at the proximal end 20. The catheter adapter 14 may include a first lumen 24 extending through the distal end 18 and the proximal end 20, and the first lumen 24 may be sealed at proximal end 20 of catheter adapter 14, such as by a split-septum connector or self-healing septum connector 25. The catheter 16 extends from the distal end 18 of catheter adapter 14 and may be configured as a PIVC that is placed into a vein of the patient, with a distal end or tip 26 of the catheter 16 positioned appropriately within the vein to enable a blood draw from the patient. Catheter 16 may be formed of any suitable material and may be of any useful length, as known to those of skill in the art.

In some non-limiting embodiments or aspects, the catheter assembly 12 may include a first fluid conduit 30 extending from the port 22. First fluid conduit 30 may be formed of any suitable material known to those of skill in the art and may have a distal end 32 and a proximal end 34. The distal end 32 of first fluid conduit 30 is coupled to port 22, while the proximal end 34 of first fluid conduit 30 may be coupled to a near-patient connector or adapter 36.

The near-patient adapter 36 may comprise a distal port 38, a proximal or “near-patient” access port 40, and a side port 42. The near-patient adapter 36 may thus be configured as a t-connector (e.g., one side port arranged at a 90 degree angle relative to a longitudinal axis of near-patient adapter 36), a y-connector (e.g., one side port arranged at a 15-85 degree angle relative to a longitudinal axis of near-patient adapter 36), or any other type of connector known in the art. In some embodiments, one or more of the ports of the near-patient adapter 36 may be configured as a luer connection, such as the proximal port 40 being configured as a female luer connection. The near-patient adapter 36 includes a lumen 44 formed therethrough having any number of branches suitable for the type of connector, such as a branch extending between the distal port 38 and proximal port 40 of the near-patient adapter 36 and a branch provided to the side port 42 of the near-patient adapter 36.

According to aspects of the disclosure, a needle-free connector 46 is coupled to or incorporated into the proximal port 40 of near-patient adapter 36. The needle-free connector 46 is configured to provide catheter access to peripheral devices, such as a blood draw device (e.g., PIVO™ from Becton, Dickinson and Company), as a non-limiting example. That is, with the near-patient adapter 36 connected to the catheter adapter 12 (via the fluid conduit 30 and side port 22), the needle-free connector 46 provides access from a peripheral device for the introduction of tubing through the indwelling catheter 16 for purposes of blood withdrawal or another fluid transfer.

In some non-limiting embodiments or aspects, catheter assembly 12 may also include an extension set 48 coupled to the side port 42 of the near-patient adapter 36. The extension set 48 may include a second fluid conduit 50 coupled to port 42 at end 52 of the conduit 50 and a far port connection 54 (e.g., needleless access connector) at opposing end 56, with a clamp 58 provided on second fluid conduit 50 that allows for occlusion thereof. The extension set 48 may be used to provide a fluid path to or from the catheter assembly 12 to enable fluid or medication delivery, blood aspiration, or connection to an ex-vivo hemodynamic monitoring device that monitors blood pressure, heart rate, and/or pulse contour of a patient, as non-limiting examples.

According to aspects of the disclosure, system 10 further includes a fluid transfer device 60 that may be operated to introduce catheter tubing (as a non-limiting example) into the vasculature of the patient, with such tubing enabling a blood draw or an administering of fluids, for example. As shown in FIG. 1 according to a non-limiting embodiment, the fluid transfer device 60 is configured as a blood draw device that includes at least a housing 62, a coupling device 64, a catheter tubing 66, and an advancement member 68. The catheter tubing 66 is moveable within the housing 62 so as to provide for advancement thereof from a first or retracted position inside the housing 62 to a second or advanced position outside of the housing 62, such that a distal end thereof may be routed into the catheter assembly 12 and into or out past the indwelling catheter 16.

According to some embodiments, the coupling device 64 is configured as a “lock” (hereafter “lock 64”) configured to physically and fluidically couple the device 60 to the catheter assembly 12. The lock 64 has a coupler 70, a blunt cannula 72, a first arm 74, and a second arm 76. In addition, the lock 64 defines a lumen (not shown) extending through the coupler 70 and the blunt cannula 72. The coupler 70 is configured to couple the lock 64 to the housing 62. The blunt cannula 72 functions as a male connection and extends from the coupler 70, with the blunt cannula disposed between the first arm 74 and the second arm 76. The blunt cannula 72 can be any suitable shape, size, and/or configuration, In some embodiments, the blunt cannula 72 can have a length that is sufficient to mate with the needle-free connector 46 and place the device 60 in fluid communication with the catheter 16 of catheter assembly 12. The blunt cannula 72 can have an inner diameter (a diameter of a surface at least partially defining the lumen therein) that is similar to or slightly larger than an outer diameter of a portion of the catheter tubing 66, so that the lock 64 can receive a portion of the catheter tubing 6 when the device 60 is transitioned between the first position and the second position.

According to other embodiments, the coupling device 64 may be configured as a male luer connector. The coupling device 64 may thus include a male protrusion having a collar formed thereabout, which may be a spin collar according to some embodiments. The male protrusion can have a length that is sufficient to mate with the needle-free connector 46 and place the device 60 in fluid communication with the catheter 16 of catheter assembly 12.

According to aspects of the disclosure, the needle-free connector 46 provided at the proximal port 40 of the near-patient adapter 36 is specifically configured to have an increased pressure rating or capability, so as to accommodate usage of the catheter assembly 12 with a fluid delivery device that provides fluid through the far port connection 54. That is, in certain operations of a fluid delivery device that injects fluid through the far port connection 54—such as when a bolus of medication is delivered at a rate higher than the catheter can accommodate and/or when the catheter is occluded—it is recognized that a high pressure may be applied against the needle-free connector 46 of near-patient adapter 36. In such cases, if the needle-free connector 46 does not have a sufficient internal pressure capability, then the needle-free connector 46 may be caused to leak responsive to such high-pressure fluid flow.

In order to prevent leakage in the needle-free connector 46, the needle-free connector 46 may be configured to include one or more pressure resistance-increasing features configured to increase a pressure capability of the needle-free connector 46. The feature(s) may improve a seal between the needle-free connector 46 and the fluid transfer device (i.e., seal with the coupling device 64) and/or provide an improved back pressure capability in the needle-free connector 46, such that the near-patient access valve or port is better able to hold higher internal pressure before leaking. In some embodiments, the needle-free connector 46 may be rated to accommodate an internal pressure of up to 300 psi.

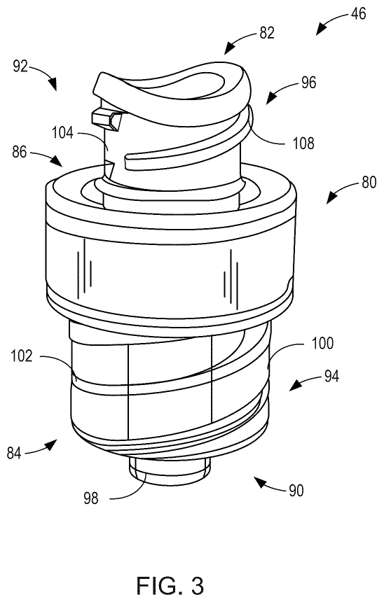

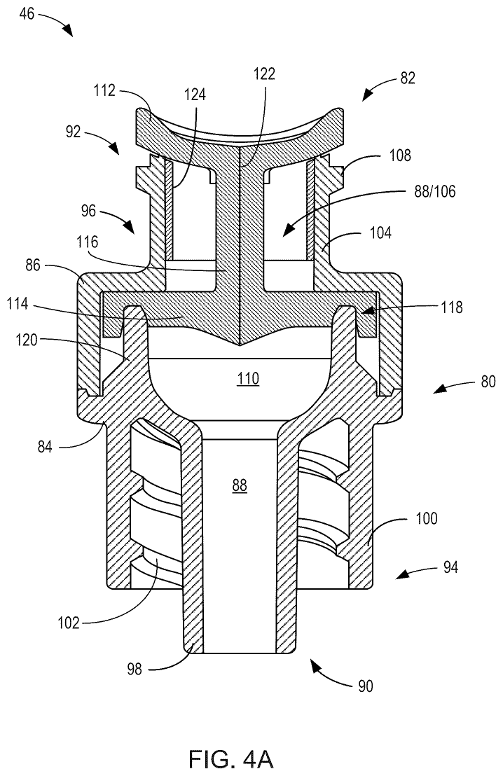

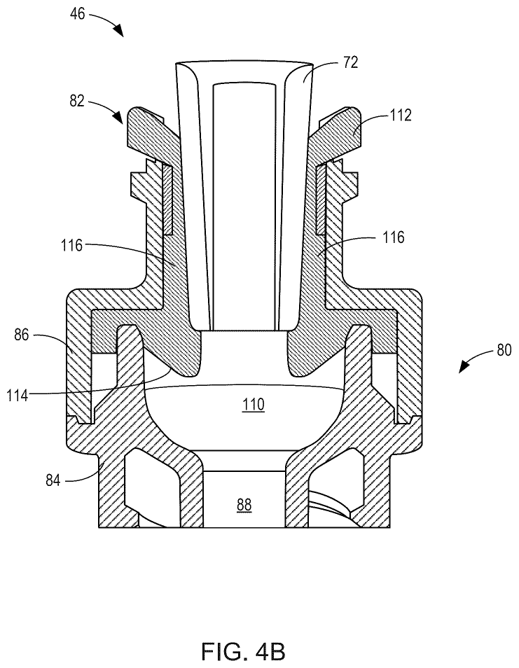

Referring now to FIG. 3 and FIGS. 4A and 4B, a needle-free connector 46 is shown according to an embodiment of the disclosure. The needle-free connector 46 may generally be described as including a main body or housing 80, a split septum valve 82 retained within the main body 80, and one or more pressure resistance-increasing features that function to increase a back-pressure capability of the connector 46, which are explained in further detail below.

The main body 80 may be constructed of a distal body portion 84 and a proximal body portion 86 that may be joined together, such as via a threaded connection, snap-fit connection, weld, adhesive, or the like. The main body 80 defines an inner lumen 88 therein that extends between a distal port 90 and a proximal port 92 of the main body 80. The main body 80 is configured such that a male luer connection 94 is provided at the distal port 90 and a female luer connection 96 is provided at the proximal port 92.

The male luer connection 94 of needle-free connector 46 includes a tapered stem or elongated member 98 having a tapered outer surface. The tapered stem 98 may be received by a corresponding tapered cavity of a female luer connection. The male luer connection 94 can also include an annular shield 100 extending about the tapered stem 98 that includes threads 102 on an inner surface thereof configured to engage corresponding threads on an outer surface of a female luer connection. As regards use of the needle-free connector 46 with the catheter assembly 12 of FIG. 1, the male luer connection 94 of needle-free connector 46 may be connected to a female luer connector of the proximal port 40 of adapter 36.

The female luer connection 96 of needle-free connector 46 includes an elongated proximal end portion 104, with the split septum valve 82 positioned over an opening of a tapered cavity 106 configured to receive and engage a corresponding tapered stem or elongated member of a male connection. The female luer connection 96 may also include an outer surface that includes threads 108 configured to engage corresponding threads on an inner surface of an annular shield of a male luer connection. As regards use of the needle-free connector 46 with the catheter assembly 12 of FIG. 1, the female luer connection 94 of needle-free connector 46 may be connected to the fluid transfer device 60 (e.g., blood draw device), such as to lock 64.

The main body 80 may define a bowl area 110 that forms part of the inner lumen 88, with the bowl being formed by the distal body portion 84. The bowl 110 may have an increased diameter as compared to a remainder of the inner lumen 88 and may be sized so as to allow for flexing/deformation of the split septum valve 82 upon engagement thereof with a male connector of an external device (e.g., the blunt cannula 72 of lock 64 on fluid transfer device 60, such as shown in FIG. 1).

As shown in FIG. 3 and FIGS. 4A and 4B, the split septum valve 82 is retained within the main body 80, such as within the elongated proximal end portion 104 and between the distal and proximal body portions 84, 86. The split septum valve 82 may generally comprise a proximal face 112, a distal face 114, and an elongated center portion 116 that extends between the two faces. The elongated center portion 116 is oriented along a longitudinal axis of the connector 46 and extends through a portion of the inner lumen 88 that is formed by proximal body portion 86. The proximal face 112 and distal face 114 of the split septum valve 82 extend radially outward from the elongated center portion 116, so as to form annular flanges at either end thereof. The proximal face 112 extends out past the elongated proximal end portion 104 of the main body 80, while the distal face 114 is positioned so as to be retained between the distal body portion 84 and the proximal body portion 86 when joined together. In some embodiments, a circumferential groove or channel 118 may be formed within a distal-facing surface of the distal face 114 that is configured to engage a protruding end 120 of the distal body portion 84, in order to better retain the distal face 114 between the distal body portion 84 and the proximal body portion 86.

A slit 122 is formed in the split septum valve 82 to selectively open or close a fluid path through the inner lumen 88 of the needle-free connector 46. The slit 122 extends a full length of the needle-free connector 46—through the proximal face 112, through the elongated center portion 116, and through the distal face 114. The slit 122 is sized to receive therein a tapered stem or elongated member of a male connection, with the male connection opening the slit 122 to create a fluid path through the inner lumen 88 of the needle-free connector 46.

According to one embodiment, and as shown in FIGS. 4A and 4B, a pressure resistance-increasing feature is provided in needle-free connector 46 in the form of one or more bump-like features 124 formed on an inner surface of the main body 80, at a location adjacent the split septum valve 82, that extend radially inward into the inner lumen 88. In an exemplary embodiment, the bump-like features 124 may be provided as a circumferential bump formed on an inner surface of the elongated proximal end portion 104 of main body 80. The circumferential bump 124 is thus aligned (in the longitudinal direction) with the elongated center portion 116 of the split septum valve 82.

As shown in FIG. 4A, the circumferential bump 124 extends radially inward from the inner surface of the elongated proximal end portion 104 of main body 80 by a distance such that a gap is provided between the circumferential bump 124 and the elongated center portion 116 of the split septum valve 82, when the split septum valve 82 is in a closed configuration (i.e., with no male connection engaged with the split septum valve 82 and the slit 122 closed). However, when a male connection 72 is engaged with the split septum valve 82 and inserted into and through the slit 122, as shown in FIG. 4B, the elongated center portion 116 of the split septum valve 82 is expanded radially outward, such that it is comes into contact with the circumferential bump 124. In this state, the circumferential bump 124 applies a radially inward-directed compressive force back against the elongated center portion 116 that prevents the elongated center portion 116 from expanding further outward. Accordingly, a tighter seal between the male connection 72 and the split septum valve 82 is maintained (as compared to if circumferential bump 124 were not present), which thereby increases a back-pressure capability of the connector 46.

In some embodiments, the configuration of the slit 122 itself functions as a pressure resistance-increasing feature for the needle-free connector 46. That is, a size of the slit 122 formed in the split septum valve 82 is reduced (as compared to the slit of needle-free connectors included in existing near port connectors), so as to form a tighter fit with a male connection 72 inserted into the split septum valve 82. According to aspects of the disclosure, in systems where the male connection engaged with the split septum valve 82 is a blunt cannula 72 (such as shown in FIG. 1), the size/length of the slit 122 is reduced to form a compressive fit with the blunt cannula 72, when engaged with the split septum valve 82. Accordingly, a tighter seal between the blunt cannula 72 and the split septum valve 82 is maintained, which thereby increases a back-pressure capability of the connector 46.

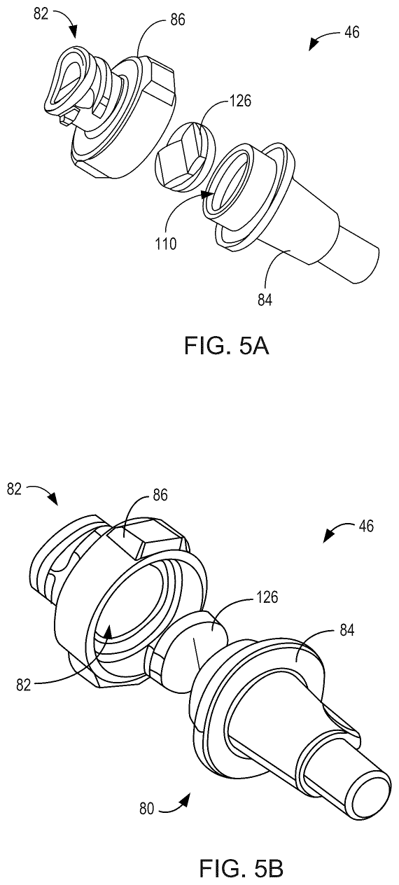

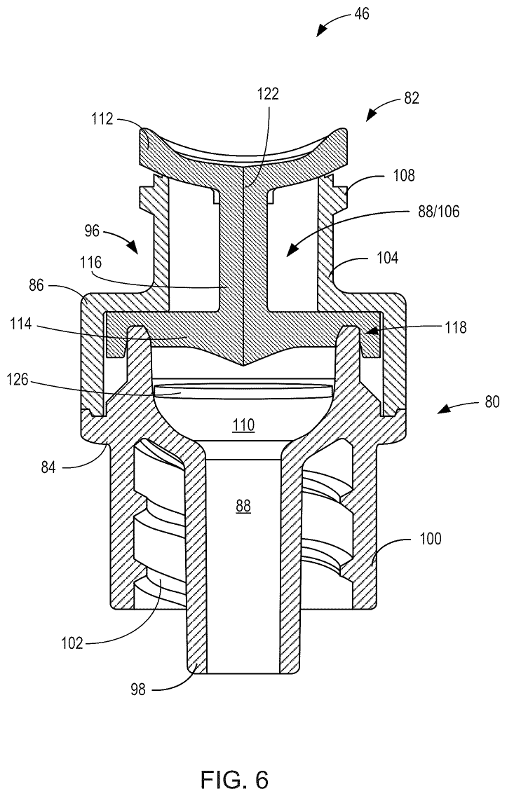

Referring now to FIGS. 5A and 5B and FIG. 6, according to another embodiment of the disclosure, a pressure resistance-increasing feature is provided in needle-free connector 46 in the form of a secondary split septum valve 126 that is included in the needle-free connector 46. The secondary split septum valve 126 may be positioned within the bowl 110 defined in/by the main body 80 (i.e., within distal body portion 84), with the bowl 110 seating and retaining the secondary valve 126 therein. With the secondary split septum valve 126 positioned within the bowl 110, the secondary split septum valve 126 is thus positioned distal to the (primary) split septum valve.

Inclusion of the secondary split septum valve 126 within needle-free connector 46 functions to increase a back-pressure capability of the connector 46, as the primary split septum valve 82 and secondary split septum valve 126 are arranged sequentially within the inner lumen 88. The arrangement of the primary split septum valve 82 and secondary split septum valve 126 enable the needle-free connector 46 to hold a higher internal pressure before leaking.

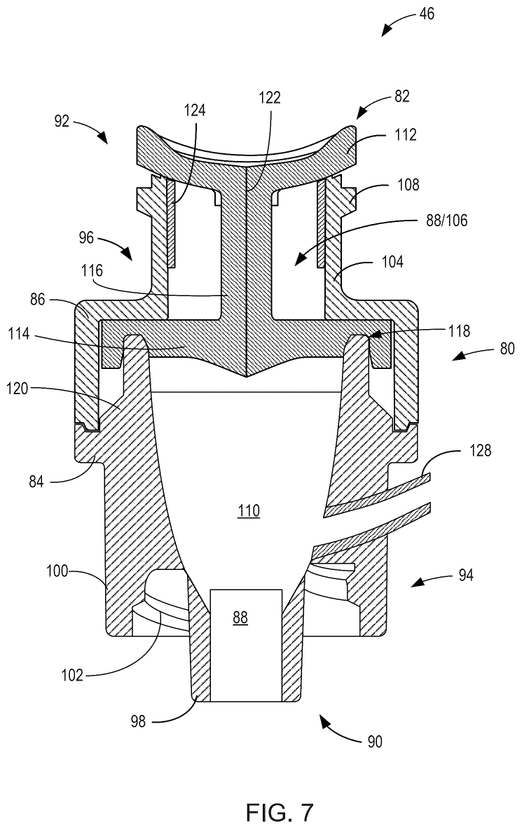

According to further embodiments of the disclosure, additional features may be added to the needle-free connector 46 to provide additional functionality thereto. As shown in FIG. 7, the needle-free connector 46 may include a side port 128 incorporated therein by which an additional connection may be made to the catheter assembly 12. In one example, an extension set (e.g., extension set 48 of FIG. 1) may be connected to the side port 128. The side port 128 may be formed in/on the main body 80 at a location between the distal port 90 and the proximal port 92, with the side port 128 in fluid communication with the inner lumen 88. The side port 128 may be configured as a (female) luer connection to/with which another external luer connection may be mated.

According to an exemplary embodiment, the side port 128 is located so as to enter into the bowl 110 distal to the split septum valve 82. In order to accommodate this positioning of side port 128, the bowl 110 may be elongated (as compared to the bowl of FIGS. 4A, 4B and 7), to provide space for the side port 128 to enter there into. With this positioning of the side port 128, the side port 128 enters into the inner lumen 88 at a point just distal to the split septum valve 82, so as to provide better flushing from the side port 128 without having to flush through the entire needle-free connector 46 to removal all residual fluids. In some embodiments, the side port 128 may be positioned so as to be offset from the centerline of the needle-free connector 46, to provide a rotational element to the flow for better flushing.

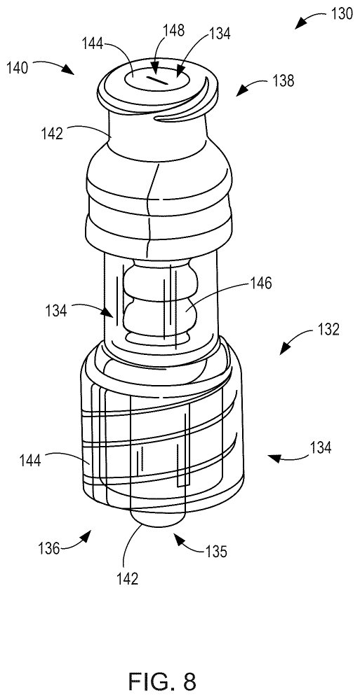

Referring now to FIG. 8, a needle-free connector 130 is shown according to another embodiment of the disclosure. The needle-free connector 130 may generally be described as including a main body or housing 132, a split septum valve 134 retained within the main body 132, and one or more pressure resistance-increasing features that function to increase a back-pressure capability of the connector 46, which are explained in further detail below.

The main body 132 may be constructed to define an inner lumen 135 therein that extends between a distal port 136 and a proximal port 140 of the main body 132. The main body 132 may be configured such that a male luer connection 134 is provided at the distal port 136 and a female luer connection 138 is provided at the proximal port 140. The male luer connection 134 at the distal port 136 includes an elongated member 142 and an annular shield 144 extending thereabout. The female luer connection 138 at the proximal port 140 includes an elongated proximal end portion 142, with the split septum valve 134 positioned over an opening of a tapered cavity configured to receive and engage a corresponding tapered stem or elongated member of a male connection.

As shown in FIG. 8, the split septum valve 134 is retained within the main body 132, such as within the elongated proximal end portion 142 and further distally into the main body 132. The split septum valve 134 may generally comprise a proximal face 144 and an elongated distal portion 146. The elongated distal portion 146 is oriented along a longitudinal axis of the connector 130 and extends through a portion of the inner lumen 135 that is formed by main body 132. A slit 148 is formed in the split septum valve 134 to selectively open or close a fluid path through the inner lumen 135 of the needle-free connector 130. The slit 148 extends through the proximal face 144 and is sized to receive therein a tapered stem or elongated member of a male connection, with the male connection opening the slit 148 to create a fluid path into the inner lumen 135 of the needle-free connector 130.

The split septum valve 134 is specifically configured to include/incorporate therein a pressure resistance-increasing feature. Specifically, the elongated distal portion 146 of the split septum valve 134 is configured as a bellows-shaped tube (or accordion-shaped tube) that extends distally from the proximal face 144 of the valve 134, with the bellows-shaped tube 146 sized and configured to receive a male connector therein. In some embodiments, the bellows-shaped tube 146 may accommodate a majority of a length of a blunt cannula therein, such as the blunt cannula 72 provided on the lock 64 of fluid transfer device 60 in FIG. 1. The bellows-shaped tube 146 may form an extended seal with the blunt cannula 72 along a length thereof that increases a back-pressure capability of the connector 46 and prevents leaks from the connector 46.

In some embodiments, a lubricant may be applied onto an internal surface of the bellows-shaped tube 146, to reduce friction during insertion of the blunt cannula 72 into the bellows-shaped tube 146. The lubricant would provide for easier insertion and mating of the blunt cannula 72 with the needle-free connector 130, without negatively affecting a seal formed between the bellows-shaped tube 146 and the blunt cannula.

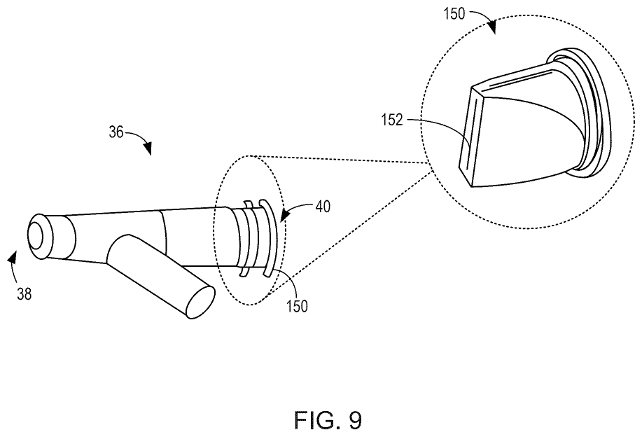

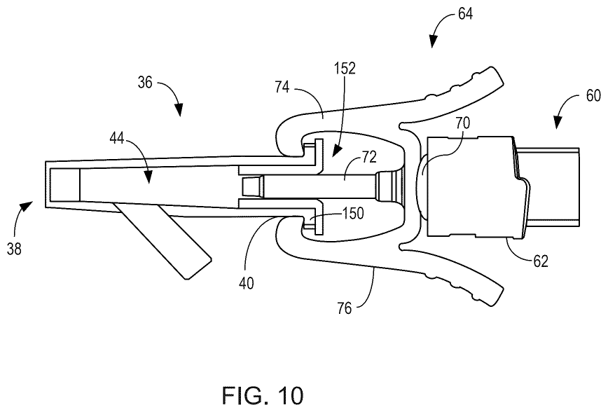

Referring now to FIGS. 9 and 10, a near-patient adapter 36 is shown that includes an access valve 150, according to another embodiment of the disclosure. The near-patient adapter 36 may have a construction as previously described-with the near-patient adapter 36 having a distal port 38, a proximal or “near-patient” access port 40, and a side port 42. In some embodiments, the proximal port 40 is configured as a female luer connection having a tapered cavity and an external thread thereon. The near-patient adapter 36 includes a lumen 44 formed therethrough having any number of branches suitable for the type of connector, such as a branch extending between the distal port 38 and proximal port 40 of the near-patient adapter 36 and a branch provided to the side port 42 of the near-patient adapter 36.

As shown in FIGS. 9 and 10, the near-patient adapter 36 includes an access valve 150 (hereafter “valve 150”) that is retained within the proximal port 40. According to non-limiting embodiments, the valve 150 may comprise a duckbill valve (as shown in FIG. 9) or, alternatively, may comprise a split septum valve or other suitable valve. The valve 150 is specifically configured to engage with a blunt cannula, such as the blunt cannula 72 provided on the lock 64 of fluid transfer device 60 in FIG. 10. That is, a sizing of the valve 150 (and of a slit or opening 152 in the valve 150) is specifically configured to receive an elongated, blunt cannula 72 to fluidly connect an external device (e.g., a blood draw or other fluid transfer device) with the inner lumen 44 of near-patient adapter 36 (and an overall catheter assembly 12). According to embodiments, the valve 150 is specifically sized/configured to receive a blunt cannula 72 having a diameter of 3 mm or less.

According to some aspects of the disclosure, with the valve 150 configured as a duckbill valve, an increasing fluid pressure on the distal/duckbill side of the valve 150 acts to apply additional pressure on the slit 152, thus helping it to seal (i.e., the pressure is working against itself to seal the slit 152). According to other aspects of the disclosure, with the valve 150 configured as a simple septum valve with a slit 152 having a decreased sized that only accommodates a blunt cannula 72, the smaller slit 152 increases the pressure capability of the valve 150—this being due to a smaller slit-to septum thickness ratio. Accordingly, a tighter seal in the valve 150 is maintained, which thereby increases a back-pressure capability of the near-patient adapter 36.

Beneficially, embodiments of the disclosure thus provide a near-patient adapter for a catheter assembly that provides an improved back-pressure capability. The near-patient adapter may include a needle-free connector that includes one or more pressure resistance-increasing features therein that are configured to increase a pressure capability of the needle-free connector. The feature(s) may improve a seal between the needle-free connector and a male connection of a fluid transfer device, such as a male luer or blunt cannula, and/or provide an improved back pressure capability in the needle-free connector, such that the needle-free connector is better able to hold a higher internal pressure before leaking.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A catheter assembly comprising:
a catheter having a catheter distal end and a catheter proximal end;
a catheter adapter coupled to the catheter proximal end and including a lumen in fluid communication with the catheter, the catheter adapter including an adapter port in fluid communication with the lumen;
a near-patient adapter comprising a distal end and a proximal end, the distal end coupled to the adapter port via an extension tube; and
a needle-free connector coupled to the proximal end of the near-patient adapter, the needle-free connector comprising:
a main body defining an inner lumen and including a distal port and a proximal port;
a split septum valve retained within the proximal port and extending into the inner lumen; and
a pressure resistance-increasing feature incorporated into or provided adjacent to the split septum valve configured, to increase a pressure capability of the needle-free connector.

2. The catheter assembly of claim 1, wherein the pressure resistance-increasing feature comprises a circumferential bump formed on an inner surface of main body and extending radially inward into the inner lumen, at a location adjacent the split septum valve.

3. The catheter assembly of claim 2, wherein the circumferential bump increases a compressive force between an external male connector and the split septum valve, when the external male connector is inserted through a slit of the split septum valve and into the inner volume of the main body.

4. The catheter assembly of claim 3, wherein the external male connector comprises one of a male luer connector and a blunt cannula.

5. The catheter assembly of claim 1, wherein the pressure resistance-increasing feature comprises a secondary split septum valve positioned within the inner lumen and distal to the split septum valve.

6. The catheter assembly of claim 1, wherein the split septum valve is configured to receive a blunt cannula of an external male connector, and wherein the pressure resistance-increasing feature comprises a reduced-length slit formed in the split septum valve.

7. The catheter assembly of claim 1, wherein the main body further includes a side port located between the distal port and the proximal port, the side port in fluid communication with the inner lumen, the side port comprising a luer connection.

8. The catheter assembly of claim 7, wherein the main body defines a bowl forming part of the inner lumen, the bowl extending distally past the split septum valve, and wherein the side port enters into the bowl distal to the split septum valve.

9. The catheter assembly of claim 7, wherein the side port is off-set from a centerline of the needle-free connector, to provide a rotational element to a flow of flushing fluid injected into the inner lumen.

10. The catheter assembly of claim 7, further comprising an extension set coupled to the side port.

11. The catheter assembly of claim 1, wherein the pressure resistance-increasing feature comprises a bellows-shaped tube provided on the split septum valve that extends distally from a slit formed in the split septum valve, the bellows-shaped tube configured to receive an external male connector therein when engaged with the split septum valve.

12. The catheter assembly of claim 1, wherein the external male connector comprises a blunt cannula, and wherein the bellows-shaped tube comprises a lubricant applied onto an internal surface thereof, to reduce friction during insertion of the blunt cannula into the bellows-shaped tube.

13. The catheter assembly of claim 1, wherein the distal port comprises a male luer connection and the proximal port comprises a female luer connection.

14. A catheter assembly useable with a fluid transfer device having a blunt cannula distal connection, the catheter assembly comprising:
a catheter having a catheter distal end and a catheter proximal end;
a catheter adapter coupled to the catheter proximal end and including a lumen in fluid communication with the catheter, the catheter adapter including an adapter port in fluid communication with the lumen;
a near-patient adapter comprising a distal end and a proximal end, the distal end coupled to the adapter port via an extension tube; and
a valve retained within the proximal end of the near-patient adapter, wherein the valve is configured to receive a blunt cannula connector of a fluid transfer device therein to fluidly connect the fluid transfer device with the inner lumen, with the blunt cannula having a diameter of 3 mm or less.

15. The catheter assembly of claim 13, wherein the valve comprises a duckbill valve.

16. The catheter assembly of claim 13, wherein the valve comprises a split septum valve.

17. A system comprising:
a catheter comprising a catheter distal end and a catheter proximal end;
a catheter adapter coupled to the catheter proximal end and including a lumen in fluid communication with the catheter, the catheter adapter including an adapter port in fluid communication with the lumen;
a near-patient adapter comprising a distal end and a proximal end, the distal end coupled to the adapter port via an extension tube;
a needle-free connector coupled to the proximal end of the near-patient adapter, the needle-free connector comprising:
a main body defining an inner lumen and including a distal port and a proximal port;
a split septum valve retained within the proximal port and extending into the inner lumen; and
a pressure resistance-increasing feature incorporated into or provided adjacent to the split septum valve configured, to increase a pressure capability of the needle-free connector; and
a blood draw device coupled to the proximal port, via the split septum valve, the blood draw device comprising a flow tube configured to be advanced distally out from the blood draw device and into or through the catheter, to enable performing of a blood draw;
wherein the blood draw device comprises a distal male connector that engages the split septum valve, with the pressure resistance-increasing feature preventing a leak out from the needle-free connector during performing of the blood draw.

18. The system of claim 16, wherein the distal male connector comprises a lock including:
a blunt cannula configured to engage the split septum valve; and
a pair of clip arms configured to secure the lock to the main body of the needle-free connector.

19. The system of claim 17, wherein the distal male connector comprises a male luer connector.

20. The system of claim 16, wherein the needle-free connector is rated to accommodate an internal pressure of up to 300 psi.
